(19) United States
(12) Patent Application Publication
(10) Pub. No.: US 2005/0080442 A1
(43) Pub. Date: Apr. 14, 2005

(54) Acupuncture needle in harmony with the golden ratio

(76) Inventor: Jack Pessin, White Plains, NY (US)

Correspondence Address: AMSTER, ROTHSTEIN & EBENSTEIN LLP, 90 PARK AVENUE, NEW YORK, NY, 10016, US

(21) Appl. No.: 10/685,023

(22) Filed: Oct. 13, 2003

(51) Int. Cl. A61B 17/34

(52) U.S. Cl. 606/189

(57) ABSTRACT

An acupuncture needle essentially in harmony with the Golden Ratio of approximately 1.618 provides the expectation and perception of certain harmonic energy-related benefits for followers of the Golden Ratio. Both the ratio of handle length to exposed needle length and the ratio of the combined handle length and exposed needle length to the handle length are substantially essentially in harmony with the Golden Ratio, while both the handle width and the needle width are essential decimal harmonics of the Golden Ratio.

### BACKGROUND OF THE INVENTION

The present invention relates to an acupuncture needle and, more particularly, an acupuncture needle in harmony with the Golden Ratio.

In a Fibonacci sequence, each number after the second is the sum of the two preceding sums: 1, 1+1=2, 1+2=3, 2+3=5, etc. When dividing the present sum by the preceding sum, one gets a ratio. The ratios converge increasingly become closer to the Golden Ratio of 1.618.

The Fibonacci sequence and the Golden Ratio (the latter being known by various names including “Golden Number,” “Divine Proportion,” and “phi” (“Φ”) appear throughout life in many biological systems such as DNA strands, nautilus shells, spiral galaxies, plants, flowers. Most importantly to acupuncture, the human body is constructed in essential respects in the Golden Ratio. Phi was first discussed by the Greeks, but no mention at all of phi is made in any classical Oriental medicine book. Those skilled in the acupuncture art will appreciate that some of the most important acupuncture points in the human body lie between the elbows and fingertips as well as between the knees and the tips of the toes. Because the bone structure of this area is constructed in the phi ratio (also common knowledge to those of skill in the medical art), it is not surprising that the most important acupuncture points lie there.

Not only are these areas in the phi ratio, but so is much of the rest of the human body. With the human body constructed in the Golden Ratio, it makes logical sense to use acupuncture needles constructed in the same ratio, especially for those who believe that there are certain energy-related properties associated with an acupuncture needle in harmony with the Golden Ratio. Surprisingly, no acupuncture text has ever mentioned the Golden Ratio.

Acupuncture needles have been known for centuries, with the first stone needles appearing between 4,000 and 10,000 years ago. Metal needles, initially made of bronze, were constructed some time later, around 800 B.C. Modem acupuncture needles, known as filiform needles, are often made of stainless steel. The needles themselves are solid, and no substances are injected or removed from the body thereby. Today, most acupuncturists use pre-packed and sterilized disposable acupuncture needles, each for use only once.

Conventional acupuncture needles are manufactured in a variety of sizes with varying ratios of handle length to needle length (i.e., the length of the needle projecting from the handle). However, no acupuncture needle has been truly constructed in essential harmony with the phi ratio. For example, an acupuncture needle by Ukiyo has a handle length of 20 millimeters and a needle length of 41 millimeters giving a handle-to-needle ratio of 0.487 and a total length (of the acupuncture needle) to needle length ratio of 1.487. Similarly, a needle by King Li has ratios of 1.07 and 2.07. Neither of these conventional acupuncture needles is essentially in harmony with the Golden Ratio of approximately 1.618.

Accordingly, it is an object of the present invention to provide an acupuncture needle substantially in essential harmony with the Golden Ratio.

### SUMMARY OF THE INVENTION

It has now been found that the above and related objects of the present invention are obtained in an acupuncture needle having a total length of about 53 millimeters (for handle and needle), a handle length of about 33 millimeters, and a needle length of about 20 mm. The ratio of the total (needle and handle) length (53 mm) to handle length (33 mm) is about 1.61, this being in essential harmony with the Golden Ratio of 1.618 to within 4/100th of a millimeter. The ratio of the handle length (33 mm) to needle length (20 mm) is about 1.6, this substantially being in essential harmony with the Golden Ratio.

In a preferred embodiment the width of the needle is about 0.16 millimeters (40 gauge), and the width of the handle is about 1.60 millimeters, both being essentially decimal harmonics of 1.618. So in multiple dimensions an acupuncture needle according to the present invention is in essential harmony with the Golden Ratio.

### BRIEF DESCRIPTION OF THE DRAWING

The above and related objects, features and advantages of the present invention will be more fully understood by reference to the following detailed description of the presently preferred, albeit illustrative, embodiments of the present invention when taken in conjunction with the accompanying drawing wherein:

FIG. 1 is a side elevational view of an acupuncture needle according to the present invention; and

FIG. 2 is an end elevational view thereof.

### DETAILED DESCRIPTION OF THE PREFERRED EMBODIMENTS

The present invention is an acupuncture needle essentially in harmony with the Golden Ratio. As used herein, the term “acupuncture needle” refers to the full length of the device, i.e., both needle and handle.

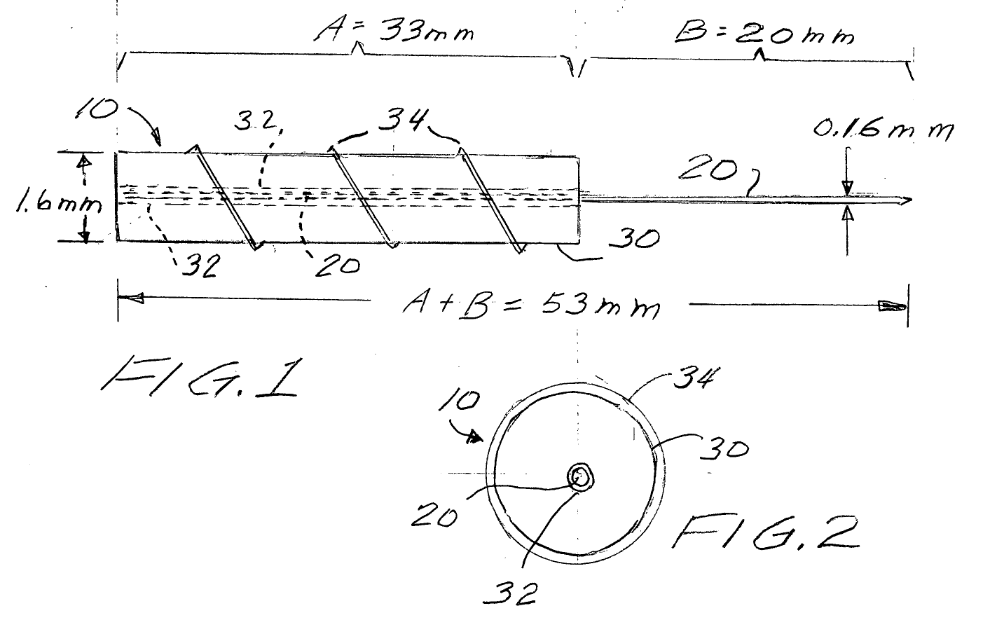

Referring now to the drawing, and in particular to FIG. 1 thereof, therein illustrated is an acupuncture needle according to the invention, generally designated by the reference numeral 10. The acupuncture needle 10 includes a needle 20 and handle 30. The handle 30 is preferably of cylindrical design and has a distal opening 32 extending from one end of (and preferably substantially throughout the length of) the handle 30 and configured and dimensioned to snugly receive a length of the needle 20. The needle 20 and handle 30 are preferably further connected by securing means (not shown) such as of an adhesive of the type well known to those skilled in the art. The outer surface of handle 30 may simply be knurled to provide a better grip but preferably defines a helix 34 or spiral. The needle may be cylindrical (as illustrated), preferably with a tapered or beveled tip, or tapered throughout its length.

Suitable materials for forming the handle 30 include, but are not limited to, stainless steel, bronze, plastic, and wood. The needle 20 is typically composed of stainless steel, but one skilled in the art will readily appreciate that other suitable metals, ceramics, or plastics may be substituted.

The length A+B of the entire acupuncture needle 10 is approximately 53 mm. The total length of the needle 20 (including the length within the handle) is preferably also approximately 53 mm., the exposed needle portion 22 (extending from the handle 30) having a length B of approximately 20 mm. The length A of handle 30 is approximately 33 mm. Thus, the ratio of handle length A plus exposed needle length B (i.e., the total length A+B of the acupuncture needle 10) to handle length A is approximately 1.61, essentially in harmony with the Golden Ratio. Further, the ratio of handle length A to exposed needle length B is approximately 1.6, substantially essentially in harmony with the Golden Ratio.

Referring now to FIG. 2 in particular, the end elevational views from both ends are substantially the same. The maximum diameter of the needle 20 is approximately 0.16 mm., while the diameter of the handle 30 is approximately 1.6 mm. These dimensions, 0.16 and 1.6, are both decimal harmonics of the Golden Ratio, thereby adding another dimension of harmony to the acupuncture needle 10 of the present invention.

The acupuncture needle of the present invention offers several benefits to the art of acupuncture. Many conventional acupuncture needles have a handle of sufficient length, but an overly-long needle length. Positioning the handle as close to the needle tip as possible provides the acupuncturist with enhanced function and comfort. Further, there are those that believe that the transmission of any signal is better with a shorter distance and thus prefer a shorter needle.

However, most importantly, there are those persons who believe that certain energy-related properties will result from an acupuncture needle essentially in harmony with the Golden Ratio. As the bone structure of the human body between the elbows and finger tips and between the knees and toes is constructed in the Golden Ratio, and as most of the important acupuncture points lie in these regions, such persons will find that an acupuncture needle essentially in harmony with the Golden Ratio will provide both the expectation and the perception of certain harmonic energy-related benefits.

To summarize, the present invention provides an acupuncture essentially in harmony with the Golden Ratio.

Now that the preferred embodiments of the present invention have been shown and described in detail, various modifications and improvements thereon will become readily apparent to those skilled in the art. Accordingly, the spirit and scope of the present invention is to be construed broadly and limited only by the appended claims, and not by the foregoing specification.

1. An acupuncture needle essentially in harmony with the Golden Ratio or Φ, comprising:
(A) a handle having a length A, and
(B) a needle having an exposed length B; whereby
(i) the ratio of the combined handle length and exposed needle length A+B to said handle length A is about 1.61; and
(ii) the ratio of said handle length A to said exposed needle length B is about 1.6.

2. The needle of claim 1 wherein said handle has a length A of about 33 mm., and said needle has an exposed length B of about 20 mm.

3. The needle of claim 1 wherein
(A) said handle has a width of about 1.6 mm., and
(B) said needle has a width of about 0.16 mm.;
whereby both said handle width and said needle width are essentially decimal harmonics of the Golden Ratio.

4. An acupuncture needle essentially in harmony with the Golden Ratio or Φ, comprising:
(A) a handle having a length of about 33 mm. and a width of about 1.6 mm.; and
(B) a needle having an exposed length of about 20 mm. and a width of about 0.16 mm.;
whereby the ratio of the combined exposed needle length and handle length to said handle length is about 1.61 and the ratio of said handle length to said exposed needle length is about 1.6; and
whereby both said handle width and said needle width are essentially decimal harmonics of the Golden Ratio.
